(19) United States
(12) Patent Application Publication
(10) Pub. No.: US 2002/0195341 A1
(43) Pub. Date: Dec. 26, 2002

(54) Alkaline pH, free solution capillary electrophoresis method

(76) Inventor: Frederic Robert, Mennecy (FR)

Correspondence Address: LERNER, DAVID, LITTENBERG,, KRUMHOLZ & MENTLIK, 600 SOUTH AVENUE WEST, WESTFIELD, NJ, 07090, US

(21) Appl. No.: 10/052,601

(22) Filed: Jan. 18, 2002

(30) Foreign Application Priority Data
Jan. 19, 2001 (FR) 01/00764

(51) Int. Cl. G01N 27/26; G01N 33/53; G01L 1/20; G01N 27/447; G01N 33/557

(52) U.S. Cl. 204/451; 435/7.1; 436/517; 204/601

(57) ABSTRACT

The invention concerns an alkaline pH, free solution capillary electrophoresis method for analysing clinical samples comprising protein constituents, characterized in that it comprises at least one step in which the sample is introduced into a capillary tube containing a buffer system comprising, as the buffer, a biological buffer with a pKa at 25° C. in the range 8.8 to 10.7 and at least one additive that can increase the ionic strength of the buffer system.

### CROSS REFERENCE TO RELATED APPLICATIONS

The present application claims priority from French Application No. 01/00764, filed Jan. 19, 2001, all of which is incorporated herein by reference.

### BACKGROUND OF THE INVENTION

The present invention relates to a process for separating proteins and peptides by capillary electrophoresis using buffer system compositions comprising an additive for use in such separation.

Blood proteins are often analysed, in particular for diagnostic purposes. The detection of monoclonal proteins can allow early diagnosis, or it can allow therapies for certain diseases to be tracked.

Proteins are routinely separated by electrophoresis, either conventional gel electrophoresis, or by capillary electrophoresis (CE). One advantage of CE is the very small quantities of sample used for analysis. Further, the use of a capillary tube with a small internal diameter to carry out electrokinetic separation disperses the heat produced by the Joule effect extremely well. This highly efficient heat dissipation associated with the high electrical resistance of capillary tubes allows high tensions to be applied, and thus produces very short separation times. Free solution CE, in which the separation medium is a simple buffer solution, is of particular application as the capillary can readily be repacked and filed with fresh solution between each analysis.

To improve the separation achieved using CE, one technique consists of using capillaries the internal surface of which has been treated. However, such coatings are not very stable and they deteriorate during use, and are thus of low reliability when carrying out a large number of analyses, which limits the advantage of the technique for routine analysis.

To analyse blood proteins using free solution CE, there is an advantage in using a buffer system with a pH of the order of 9 to 11, preferably about 10.

Alkaline buffer systems include borate buffers such as those described in U.S. Pat. No. 5,120,413. Such buffers form complexes with glycoproteins. Most blood proteins are glycosylated. The formation of such complexes modifies the electrophoretic mobility of glycoproteins. With such a borate buffer, at a pH of about 10, blood proteins are usually divided into 6 fractions (gamma, beta-2, beta-1, alpha-2, alpha-1, albumin). There is a risk that some monoclonal proteins will co-migrate with normal protein fractions, and during analysis, certain normal protein fractions may mask certain monoclonal proteins.

As an example, analysing serums containing monoclonal proteins, in particular certain proteins of the IgM kappa type, shows that the corresponding peak co-migrates with one of the protein fractions (the beta-2 fraction), resulting in a high risk of non detection of such IgM kappa.

### SUMMARY OF THE INVENTION

The Applicant has now demonstrated that rapid and efficient protein separation can be carried out using free solution CE employing an alkaline buffer as the buffer system, i.e., with a pH of 9 to 11, more precisely about 10, comprising a zwitterionic biological buffer as the buffer and in addition, at least one additive that can increase the ionic strength of the buffer system.

The present invention provides alkaline pH, free solution capillary electrophoresis method for analysing clinical samples comprising protein constituents, this method comprising at least one step in which the sample is introduced into a capillary tube containing a buffer system comprising, as the buffer, a biological buffer with a pKa at 25° C. in the range 8.8 to 10.7 and at least one additive that can increase the ionic strength of the buffer system.

The Applicant has demonstrated that select a combination of a zwitterionic biological buffer with an alkaline pH of the order of 9 to 11, more precisely about 10, and an additive that can increase the ionic strength of the electrophoresis medium can achieve improved separation.

The separations are reproducible. Further, the zwitterion has a majority of negative charges at a pH of about 10, which may be advantageous within the context of the invention.

Finally, as will become apparent from the examples, certain proteins appear and can be detected in the form of a peak that is more distinct or clearly separated from other fractions when compared with the detection achieved using a borate buffer.

As will also become apparent in the examples, separations carried out using the buffers of the present invention can produce a separation that is equivalent to or identical to that which can be observed with other techniques, in particular gel electrophoresis, and with a greater accuracy, reproducibility and resolution.

Other characteristics and advantages of the invention will become apparent from the following detailed description made with reference to the accompanying drawings.

### BRIEF DESCRIPTION OF THE DRAWINGS

FIGS. 1A and 1B show electropherograms obtained by capillary electrophoresis using buffer systems of the invention.

FIGS. 1C and 1D respectively show the gel and the densitometric profile of the same serum using gel electrophoresis.

FIG. 2A shows an electropherogram of a serum with a monoclonal gammapathy, analysed by capillary electrophoresis using a buffer system of the invention.

FIGS. 2B and 2C respectively show the gel and the densitometric profile of the same serum obtained with gel electrophoresis.

FIG. 3A shows an electropherogram of a serum with biclonal gammapathy, analysed by capillary electrophoresis using a buffer of the invention.

FIGS. 3B and 3C respectively show the gel and densitometric profile of the same serum carried out by gel electrophoresis.

FIG. 4A shows an electropherogram of a serum with low monoclonal gammapathy analysed by capillary electrophoresis using a buffer of the invention.

FIGS. 4B and 4C respectively show the gel and the densitometric profile of the same serum carried out by gel electrophoresis.

FIG. 5A shows an electropherogram of a serum comprising a monoclonal protein migrating at the limit of the γ and β fractions, analysed by capillary electrophoresis using a buffer of the invention.

FIGS. 5B and 5C respectively show the gel and the densitometric profile of the same serum carried out by gel electrophoresis.

FIG. 5D shows an electropherogram of the same serum analysed by capillary electrophoresis using the usual borate buffer.

FIG. 6 shows ten electropherograms, numbered 1 to 10, of the same serum obtained during 10 successive analyses using capillary electrophoresis employing a buffer of the invention.

### DETAILED DESCRIPTION

In accordance with the present invention, the buffer system can be any normal known buffer known as a biological buffer and having a pKa at 25° C. in the range 8.8 to 10.7, i.e., compatible with in vivo applications, adapted to the desired separation, and useful for electrophoresis in general, and in particular for capillary electrophoresis. Preferably, biological buffers with a high buffering power with a pH of about 10 are selected.

Amongst the biological buffers useful according to the invention, a buffer of the Good type is particularly cited as the CAPS defined herebelow and analogs. The Good type buffers according to the invention are zwitterionic and have a pKa at 25° C. between 8.8 and 10.7. The buffer of the Good type and their analogs comprise amine and acid functions.

Particularly biological buffers suitable according to the invention that can be cited are AMPD (2-amino-2-methyl-1,3-propanediol), TABS (N-tris[hydroxymethyl]methyl-4-aminobutanesulphonic acid), AMPSO (3-[(1,1-dimethyl-2-hydroxyethyl)amino]-2-hydroxypropanesulphonic acid), CHES (2-(N-cyclohexylamino)ethanesulphonic acid), CAPSO (3-[cyclohexylamino]-2-hydroxy-1-propanesulphonic acid), AMP (2-amino-2-methyl-1-propanol), CAPS (3-cyclohexylamino-1-propanesulphonic acid) and CABS (4-[cyclohexylamino]-1-butanesulphonic acid) and mixtures thereof. Other zwitterionic biological buffers can be used in the invention. The amino acid buffers are however not intended as a buffer or additive according to the present invention.

In accordance with the invention, AMPD, TABS, AMPSO, CAPSO, AMP, CAPS and CABS buffers are preferred. More preferably, CAPS, CAPSO or CABS are used. More particularly preferably, CAPS is used.

Compounds that can be cited for use as the additive to the buffer for use in accordance with the invention that can increase the ionic strength of the electrolyte are selected from alkali metal chlorides, sulphates, sulphonates, carbonates, carboxylates, fluorides and phosphates and mixtures thereof. Of these, alkali metal chlorides, sulphates and sulphonates and mixtures thereof are preferred.

More preferably, the sulphate is used.

Preferably, sodium or potassium salts are selected.

Of the additives cited above, sodium sulphate is preferred.

Preferably, in accordance with the invention, CAPS is associated with sodium sulphate.

These compounds are known per se and are commercially available.

The term “sample in accordance with the invention” means the biological sample to be analysed, diluted with a suitable diluting solution or buffer system, for example, or pure, which is analysed with the buffer system, i.e., the electrolyte, for example by introducing the sample into a capillary filled with that buffer.

The clinical sample for analysis and the term “clinical sample” as used here means any biological liquid from healthy humans or human patients. The human biological liquids can be normal or diseased serum, and also haemolysed serum, plasma, urine, or cerebro-spinal fluid. The processes and compositions according to the present invention are particularly useful for the analysis of serum, plasma, urine, or cerebro-spinal fluid.

The samples can also be synthetic proteins, and the method of the invention can, for example, be intended for production control.

The method of the invention is of particular application in analysing serum, and for separating blood proteins.

In blood samples, the blood proteins to be separated are primarily albumin and the α₁; α₂; β (or β₁ and β₂); and γ globulin fractions.

The pH of the buffer of the invention, i.e., the pH of the biological buffer with the additive, can be between 9 and 11, particularly preferably about 10.

The buffer systems of the invention can also comprise at least one pH-modifying component. The pH-modifying compound can be a compound selected from lithium hydroxide, sodium hydroxide, potassium hydroxide, rubidium hydroxide, caesium hydroxide, francium hydroxide, or a mono-, di-, tri- or tetra-alkyl ammonium hydroxide containing 1 to 8 carbon atoms in the alkyl portion.

In accordance with the invention, the biological buffers are used under the usual conditions, at concentrations in the buffer system of the order of 10 to 500 mM, preferably more than 20 and less than 200 mM.

The salts used as additives in accordance with the invention are used at concentrations in the buffer system of 10 mM to 500 mM, preferably 50 to 200 mM, more preferably about 150 mM.

The buffer systems of the invention can also comprise at least one additive comprising a negatively charged anionic pole at a pH of more than 9, and a hydrophobic portion, in particular C₆ to C₂₂ alkyl-mono-, di- or tri-sulphonates, C₆ to C₂₂ alkylmono-, di- or tri-carboxylates, C₆ to C₂₂ alkylcarboxysulphonates, and in particular C₆ to C₁₀ alkylsulphonates.

The above di- and tri-carboxylates, di- and tri-sulphonates and carboxysulphonates are thus combinations of one or more carboxylate or sulphonate functions on C₆ to C₂₂ alkyl chains. Non limitative examples thereof arc the 1,2,3-nonadecanetricarboxylic acid (three carboxylate functions and a en C₁₉ alkyl chain), the 2-methyl-2-sulfooctadecanoic acid (one carboxylate function and one sulfonate function and a C₁₈ alkyl chain) and the 1,12-dodecanedicarboxylic acid (two carboxylate functions and a C₁₂ alkyl chain).

Preferably, octanesulphonate is used, in concentrations of the order of 1 to 5 mM, preferably 1 to 4 mM and more preferably 2.5 mM.

The buffer compositions of the invention are prepared in a manner that is normal when preparing buffer system compositions, namely by adding the constituents in the liquid form, or as a solid to be diluted, to an acceptable support. Usually, the support is water, either distilled or demineralised.

The materials used for the capillaries are those routinely employed in capillary electrophoresis. It is possible to use fused silica capillaries with an internal diameter of 5 to 200 μm. Preferably, capillaries with an internal diameter of less than 100 μm are used; more preferably, less than 50 μm. Preferably, capillaries with an untreated internal surface are used. The skilled person will be capable of adapting the nature and size of the capillary to the analytical requirements.

### EXAMPLES

### Materials and Methods

A) Capillary Electrophoresis (Method A)

Capillary electrophoresis was carried out on clinical samples using a CE apparatus provided with a fused silica capillary with an internal diameter of 25 microns. Detection was carried out at 200 nm. The samples were placed in the apparatus's sample changer and automatically injected by hydrodynamic injection (50 mbars for 7 s). The samples were separated within 10 minutes by applying an electrical field of about 400 V/cm. The capillary was washed with 0.5 M sodium hydroxide before each analysis, then with the buffer system.

Buffer systems:

Analytical grade chemical substances were used.

A first buffer in accordance with the invention was prepared by dissolving 11.07 g of CAPS (molar mass 221.3 g/mole) and 21.3 g of sodium sulphate (molar mass 142.04 g/mole) in 1 litre (1) of demineralised water. The final concentration was 50 mM of CAPS and 150 mM of sodium sulphate, and the pH was adjusted to 10.0 by adding sodium hydroxide pellets (molar mass: 40.0 g/mole).

A second, preferred, buffer system was prepared as described above, adding octanesulphonate in a concentration of 2.5 mM.

Electrophoresis carried out using method A above with the CAPS/sodium sulphate buffer produced a protein profile with 5 fractions, the gamma, beta, alpha-2, alpha-1 and albumin fractions, reading from left to right.

The borate buffer was prepared by dissolving 9.3 g of boric acid (molar mass: 61.83 g/mole) in 11 of demineralised water, and 5.1 g of sodium hydroxide (molar mass: 40.0 g/mole). The final concentration was 150 mM and the pH was 10.0.

Electrophoresis carried out using method A above with the borate buffer produced a protein profile with 6 fractions, the gamma; beta-2, beta-1, alpha-2, alpha-1 and albumin fractions, reading from left to right.

B) Agarose Gel Electrophoresis (Method B)

Agarose gel was used to carry out a comparative analysis of the blood proteins. 10 μl of serum was loaded into each well in the membrane applicator described in European patent EP-A-0 493 996, U.S. Pat. No. 5,464,515 and U.S. Pat. No. 5,405,516. The loaded applicator was then applied to the surface of an agarose gel for 30 seconds. The samples applied to this gel were separated by electrophoresis for about 7.5 minutes at a power of 20 W, using an instrument that could regulate the temperature to 20° C. After migration, the gel was dried and stained with acid black. After staining, the gel was decolorised and dried again. The gels were then analysed by densitometry to produce the protein profiles.

For a normal serum, a protein profile was obtained with 5 fractions, the gamma, beta, alpha-2, alpha-1 and albumin fractions, reading from left to right.

C) Clinical Samples:

For the CE, the human aenim was diluted to 1/10^{th} in the buffer system.

### Example 1

With the first and second buffers described above, normal serum was analysed.

Electrophoresis was carried out using method A above.

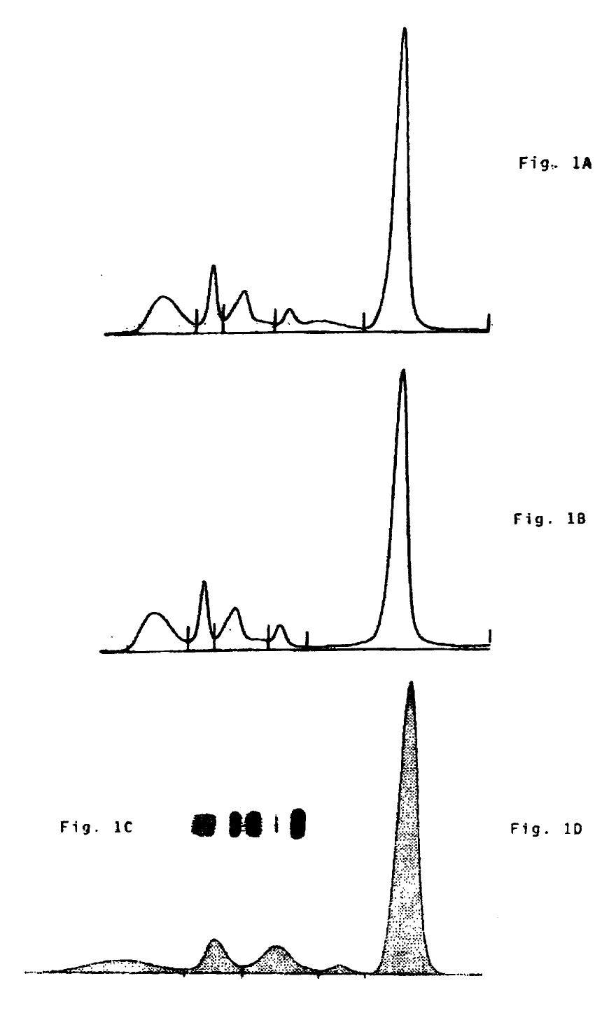

As can be seen from FIGS. 1A and 1B, the electropherograms obtained exhibited five peaks, successively attributed to γ, β, α₂, α₁ globulin and albumin, reading from left to right.

### Example 2 (Comparative)

The protein profile (the gel in FIG. 1C and its densitometric profile of FIG. 1D) was obtained by analysing the same serum as in the preceding example using method B above. As can be seen from these figures, the protein profile obtained exhibited 5 fractions, γ, β, α₁, α₂ and albumin, reading from left to right. Comparison with the result obtained in Example 1 shows that the implementations of the invention can produce a protein profile with 5 fractions comparable with that obtained with agarose gel.

### Example 3

The second buffer described above was used to analyse a serum with monoclonal gammapathy.

The electrophoresis was carried out as described in Example 1.

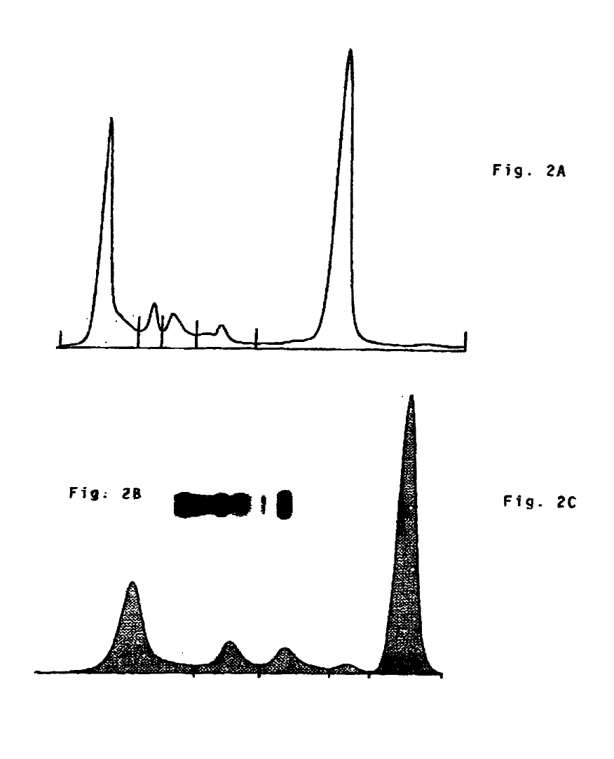

As can be seen in FIG. 2A, the electropherogram obtained exhibits five successive peaks, attributed to γ, β, α₂, α₁ globulin and albumin fractions respectively. Note the presence of a supplemental peak in the gamma fraction, corresponding to the monoclonal protein present in the analysed serum.

### Example 4 (Comparative)

The protein profile (the gel in FIG. 2B and its densitometric profile of FIG. 2C) was obtained by analysing the same serum as in the preceding example using method B above. Comparison with the result obtained in Example 3 shows that the implementations of the invention can achieve a resolution equivalent to that obtained with agarose gel.

### Example 5

The second buffer described above was used to analyse a serum with biclonal gammapathy.

The electrophoresis was carried out as described in Example 1.

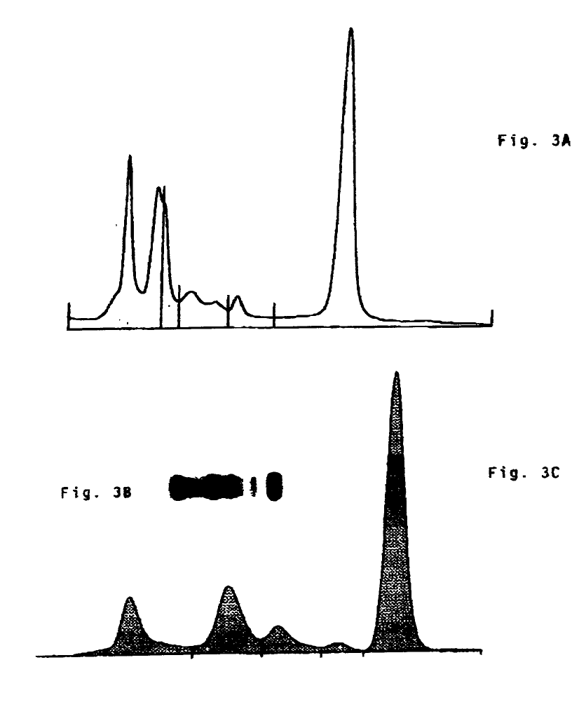

As can be seen in FIG. 3A, the electropherogram obtained showed two supplemental peaks in the gamma fraction, corresponding to the two monoclonal proteins present in the analysed serum.

### Example 6 (Comparative)

The protein profile (the gel in FIG. 3B and its densitometric profile of FIG. 3C) was obtained by analysing the same serum as in the preceding example using method B above. Comparison with the result obtained in Example 5 showed that the implementation of the invention can produce a resolution that is higher than the resolution obtained with an agarose gel. On agarose gel one of the monoclonal protein co-migrated with the beta fraction.

### Example 7

The second buffer described above was used to analyse a serum with weak monoclonal gammapathy.

The electrophoresis was carried out as described in Example 1.

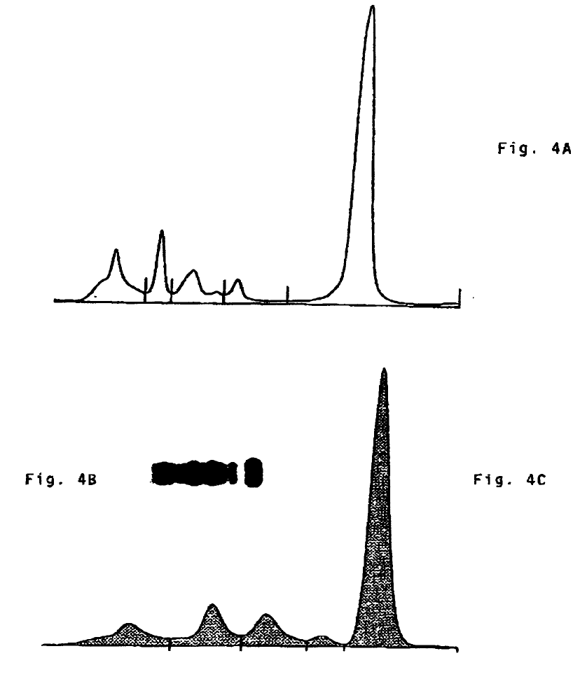

As can be seen in FIG. 4A, the electropherogram obtained showed a small supplemental peak in the gamma fraction, corresponding to the monoclonal protein present in the analysed serum.

### Example 8 (Comparative)

The protein profile (the gel in FIG. 4B and its densitometric profile of FIG. 4C) was obtained by analysing the same serum as in the preceding example using method B above. Comparison with the result obtained in Example 7 showed that the implementation of the invention can achieve a sensitivity substantially identical to that obtained with agarose gel.

### Example 9

The second buffer described above was used to analyse a serum with a monoclonal protein of the IgM kappa type migrating to the limit of the γ and β fractions.

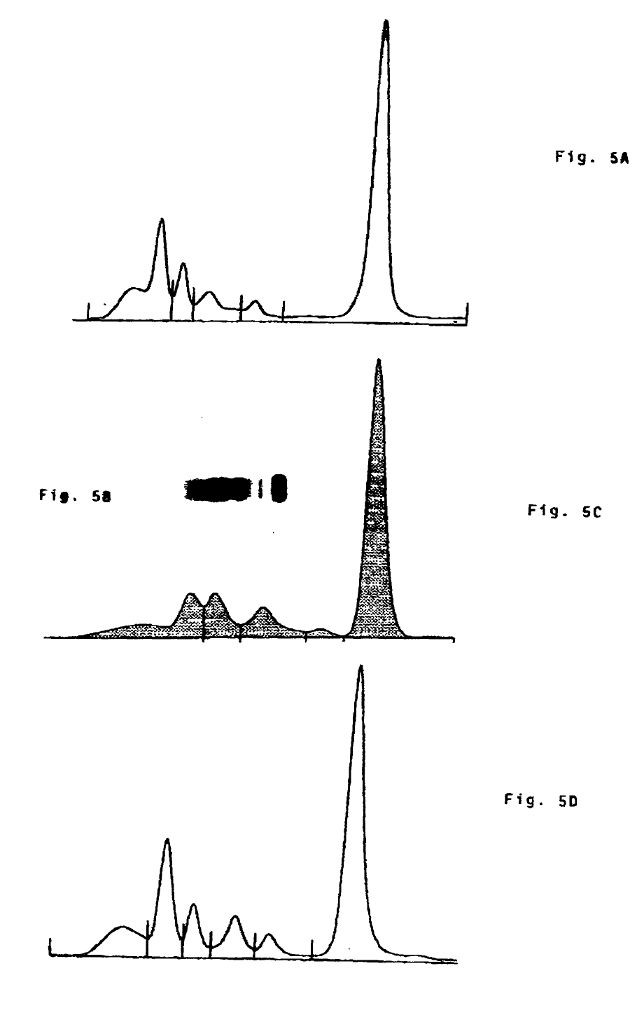

As can be seen in FIG. 5A, the electropherogram obtained showed a supplemental peak in the gamma fraction, corresponding to the monoclonal protein present in the analysed serum.

### Example 10 (Comparative)

The protein profile (the gel in FIG. 5B and its densitometric profile of FIG. 5C) was obtained by analysing the same serum as in the preceding example using method B above. Comparison with the result obtained in Example 9 showed that the implementation of the invention can achieve a detection that is substantially identical to that obtained with agarose gel.

### Example 11 (Comparative)

The procedure of Example 1 was followed, the buffer system used being the normal borate buffer prepared as indicated above.

Electrophoresis was carried out using method A above.

As can be seen in FIG. 5D, the electropherogram obtained exhibited six successive peaks, attributed respectively to the γ, β₂, β₁, α₂, α₁ globulin and albumin fractions, reading from left to right.

No perturbation in the protein profile was observed with this borate buffer, only an increase in the percentage of the beta fraction above normal values that could give rise to suspecting the presence of a monoclonal protein in this serum; comparison with the result obtained in Example 9 shows that this implementation of the invention can achieve a higher resolution compared with that obtained with CE using the normal borate buffer for certain kappa IgM type monoclonal proteins.

### Example 12

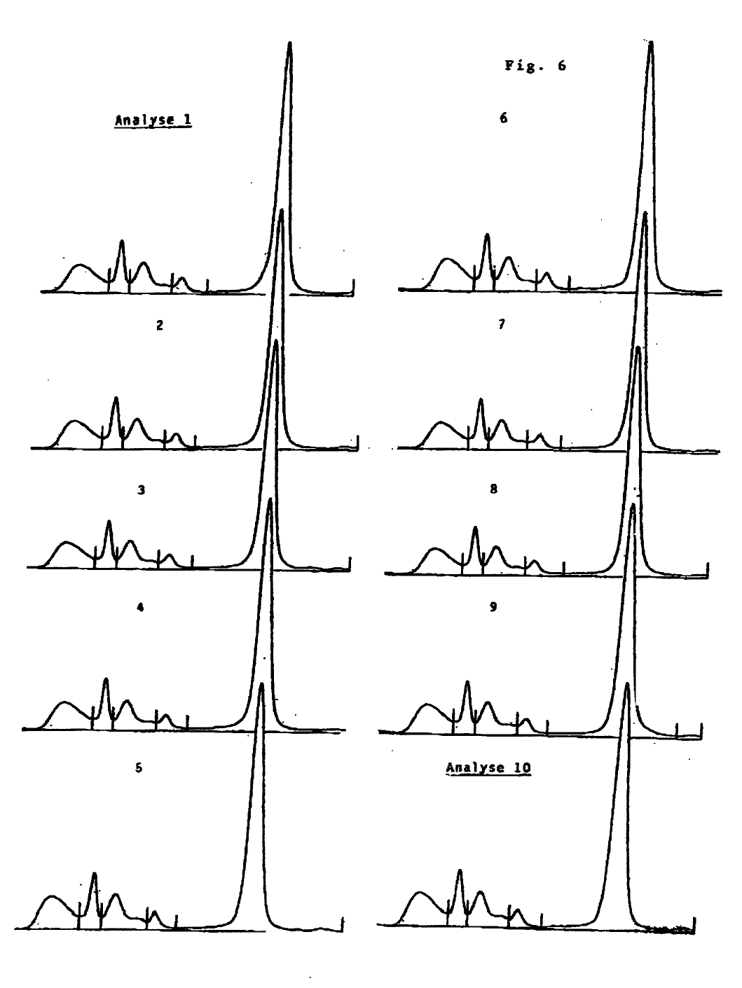

Using the second preferred buffer, 10 consecutive analyses were carried out on the same serum by capillary electrophoresis using method A described above; as can be seen from FIG. 6, the reproducibility of the profiles was excellent.

1. An alkaline pH, free solution capillary electrophoresis method for analyzing a clinical sample comprising protein constituents said method comprising: introducing said clinical sample into a capillary tube containing a buffer system wherein said buffer system comprises a biological buffer with a pKa at 25° C. in the range 8.8 to 10.7 and at least one additive that increases the ionic strength of said buffer system.

2. The method of claim 1, which further comprises separating said protein constituents by migration and detecting said protein constituents.

3. The method of claim 1, wherein the clinical sample is serum, plasma, hemolyzed blood, urine or cerebrospinal fluid.

4. The method of claim 1, wherein said protein constituents are blood proteins.

5. The method of claim 1, wherein said protein constituents are selected from albumin or α₁-globulin, α₂-globulin, β-globulin, β₁-globulin, β₂-globulin and γ-globulin.

6. The method of claim 1, wherein biological buffer is selected from 2-amino-2-methyl-1,3-propanediol (AMPD), N-tris(hydroxymethyl)methyl-4-aminobutanesulphonic acid (TABS), 3-[(1,1-dimethyl-2-hydroxyethyl)amino]-hydroxypropanesulphonic acid (AMPSO), 2-(N-cyclohexylamino)ethanesulphonic acid (CHES), 3-(cyclohexylamino)-2-hydroxy-1-propanesulphonic acid (CAPSO), 2-amino-2-methyl-1-propanol (AMP), 3-cyclohexylamino-1-propanesulphonic acid (CAPS) and 4-(cyclohexylamino)-1-butanesulphonic acid (CABS).

7. The method of claim 1, wherein the biological buffer is selected from 2-amino-2-methyl-1,3-propanediol (AMPD), N-tris(hydroxymethyl)methyl-4-aminobutanesulphonic acid (TABS), 3-[(1,1-dimethyl-2-hydroxyethyl)amino]-hydroxypropanesulphonic acid (AMPSO), 2-(N-cyclohexylamino)ethanesulphonic acid (CHES), 3-(cyclohexylamino)-2-hydroxy-1-propanesulphonic acid (CAPSO), 2-amino-2-methyl-1-propanol (AMP), 3-cyclohexylamino-1-propanesulphonic acid (CAPS) and 4-(cyclohexylamino)-1-butanesulphonic acid (CABS).

8. The method of claim 1, wherein the biological buffer is selected from 3-cyclohexylamino-1-propanesulphonic acid (CAPS), 3-(cyclohexylamino)-2-hydroxy-1-propanesulphonic acid (CAPSO) and 4-(cyclohexyamino)-1-butanesulphonic acid (CABS).

9. The method of claim 1, wherein the biological buffer is 3-cyclohexylamino-1-propanesulphonic acid (CAPS).

10. The method of claim 1, wherein said biological buffer in the buffer system has a concentration in the range of 10 to 500 mM.

11. The method of claim 1, wherein said biological buffer in said buffer system has a concentration of more than 20 and less than 200 mM.

12. The method of claim 1, wherein said additive that increases the ionic strength of said buffer system is selected from alkali metal chlorides, sulphates, sulphonates, carboxylates, fluorides, carbonates, phosphates, and mixtures thereof.

13. The method of claim 1, wherein said additive that increases the ionic strength of said buffer system is selected from alkali metal chlorides, sulphates, sulphonates, carboxylates, orides, and mixtures thereof.

14. The method of claim 1, wherein said additive that increases the ionic strength of said buffer system is a chloride, sulphate or sulphonate.

15. The method of claim 1, wherein the additive that increases the ionic strength of said buffer system is sodium sulphate.

16. The method of claim 1, wherein said additive that increases the ionic strength of said buffer system and has a concentration in the range of 10 to 500 mM.

17. The method according to claim 1, wherein said additive increases the ionic strength of an electrolyte and has a concentration of more than 50 and less than 200 mM.

18. The method according to claim 1, wherein said buffer system further comprises at least one buffer component selected from C₆ to C₂₂ alkyl-mono-, di- or tri-sulphonates, C₆ to C₂₂ alkylmono-, di- or tri-carboxylates, and C₆ to C₂₂ alkylcarboxysulphonates.

19. The method according to claim 1, wherein said buffer system further comprises a C₆ to C₁ alkylsulphonate.

20. The method according to claim 1, where said buffer system further comprises octanesulphonate.

21. The method according to claim 19, wherein said alkylsulphonate has a concentration in the range 1 to 5 mM.

22. The method according claim 1, wherein said biological buffer has a pH in the range 9 to 11.

23. The method according claim 22, wherein the pH of said buffer is about 10.

24. The method according to claim 1, wherein the capillary tube is produced from fused silica.

25. The method according to claim 1, wherein said buffer system ether comprises at least one pH-modifier.
